(19) United States
(12) Patent Application Publication
(10) Pub. No.: US 2010/0056905 A1
(43) Pub. Date: Mar. 4, 2010

(54) SYSTEM AND METHOD FOR TRACKING MEDICAL DEVICE

(75) Inventor: Peter Anderson, Andover, MA (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/204,384

(22) Filed: Sep. 4, 2008

(51) Int. Cl. A61B 6/00

(52) U.S. Cl. 600/424; 378/4

(57) ABSTRACT

In one embodiment, a method for electromagnetic tracking is provided. The method comprises mounting at least one receiver coil array on each of a plurality of primary distortion sources, selecting one of the primary distortion source as a secondary distortion source, acquiring mutual inductance signals between a transmitter coil array and the secondary distortion source, the transmitter coil array being rigidly attached to a surgical tool, acquiring mutual inductance signals between the transmitter coil array and at least one primary distortion source, estimating an initial position for the surgical tool in the presence of the primary distortion source and the secondary distortion source, refining the estimated position of the surgical tool and estimating an orientation of the surgical tool.

### FIELD OF INVENTION

The invention generally relates to a system and method for determining the position and orientation of a remote device relative to a reference coordinate frame using magnetic fields and more particularly to a system and method for determining the position and orientation of a medical device, such as a catheter, within a patient.

### BACKGROUND OF THE INVENTION

Electromagnetic trackers are sensitive to conductive or ferromagnetic objects. Presence of metallic targets near to an electromagnetic transmitter (Tx) or an electromagnetic receiver (Rx) may distort transmitting signals resulting in inaccurate position and orientation (P&O) measurement. Further, X-ray detectors and X-ray sources are fixedly present in the imaging room adding to the distortion of the transmitting signals.

Accordingly, it would be desirable to provide a tracking system of enhanced accuracy having enhanced immunity to common field distortions caused by X-ray detectors and X-ray sources.

### BRIEF DESCRIPTION OF THE INVENTION

The above-mentioned shortcomings, disadvantages and problems are addressed herein which will be understood by reading and understanding the following specification.

In one embodiment, an intra-operative imaging and tracking system for guiding a surgical tool during a surgical procedure performed on a patient is provided. The intra-operative imaging and tracking system comprises a fluoroscope having an X-ray source, an X-ray detector and a support structure configured to support the X-ray source and the X ray detector, the X-ray source and the X-ray detector being movable about the patient to generate a plurality of two-dimensional X-ray images of the patient from different views, a tracking system comprising a transmitter coil array configured to generate an electromagnetic field in an area of interest, the transmitter coil array being affixed to the surgical tool and at least one receiver coil array configured to generate a sensing signal in response to sensed electromagnetic field, the at least one receiver coil array being secured against movement relative to one of a plurality of primary distortion sources, a signal measuring circuit electrically coupled to the tracking system to measure generated and sensed signals to form a matrix representing mutual inductance between the transmitter coil array and the receiver coil array, a processor operative with the mutual inductance matrix and the X-ray images to determine coordinates of the transmitter coil array affixed to the surgical tool and position of the surgical tool relative to the patient.

In another embodiment, a method for electromagnetic tracking is provided. The method comprises mounting at least one receiver coil array on each of a plurality of primary distortion sources, selecting one of the primary distortion source as a secondary distortion source, acquiring mutual inductance signals between a transmitter coil array and the secondary distortion source, the transmitter coil array being rigidly attached to a surgical tool, acquiring mutual inductance signals between the transmitter coil array and the at least one primary distortion source, estimating an initial position for the surgical tool in the presence of the primary distortion source and the secondary distortion source, refining the estimated position of the surgical tool and estimating an orientation of the surgical tool.

In yet another embodiment, a computer-readable media having computer-executable instructions thereon that, when executed by a computer, perform a method for electromagnetic tracking is provided. The method comprises mounting at least one receiver coil array on each of a plurality of primary distortion sources; selecting one of the primary distortion source as a secondary distortion source, acquiring mutual inductance signals between a transmitter coil array and the secondary distortion source, the transmitter coil array being rigidly attached to a surgical tool, acquiring mutual inductance signals between the transmitter coil array and the at least one primary distortion source, estimating an initial position for the surgical tool in the presence of the primary distortion source and the secondary distortion source, refining the estimated position of the surgical tool and estimating an orientation of the surgical tool.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a block diagram of an intra-operative imaging and tracking system, in an embodiment; and

FIG. 2 shows a flow diagram of a method of electromagnetic tracking of a medical device, in another embodiment.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

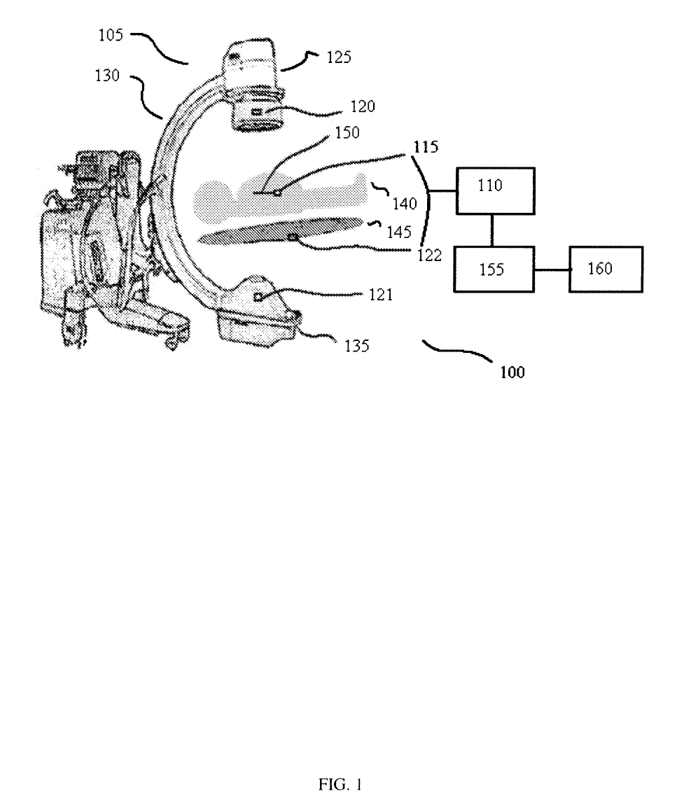

FIG. 1 illustrates an intra-operative imaging and tracking system 100 for use in surgical navigation, in an operating room or clinical setting, to determine the position and orientation of a medical device, such as a guide wire, catheter, implant, surgical tool, marker or the like. As shown, the system 100 includes a fluoroscope 105 and a tracking system 110. The tracking system 110 comprises a transmitter coil array 115 and a plurality of receiver coil arrays 120, 121 and 122. The fluoroscope 105 is illustrated as a C-arm fluoroscope 105 in which an X-ray source 125 is mounted on a structural member or C-arm 130 opposite to an X-ray detector 135. The C-arm 130 moves about a patient 140 for producing two dimensional projection images of the patient 140 from different angles. The patient 140 remains positioned between the X-ray source 125 and the X-ray detector 135, and may, for example, be situated on a table 145 or other support. In the illustrated system 100, the transmitter coil array 115 is affixed to, incorporated in or otherwise secured against movement with respect to a surgical tool 150 or probe. One of the receiver coil array 120 is fixed on or in relation to the X-ray source 125, a second receiver coil array 121 is fixed on or in relation to the X-ray detector 135 and a third receiver coil array 122 is fixed on or in relation to the patient support 145. The surgical tool 150 may be a rigid probe as shown in FIG. 1, allowing the transmitter coil array 115 to be fixed at any known or convenient position, such as on its handle, or the surgical tool 150 may be a flexible tool, such as a catheter, flexible endoscope or an articulated tool. In the latter cases, the transmitter coil array 115 may be a small, localized element positioned in or at the operative tip of the surgical tool 150 to track coordinates of the tip within the body of the patient 140.

The electromagnetic tracking system 110 typically employs ISCA (Industry-standard Coil Architecture) 6-DOF (6 Degrees of Freedom) tracking technology. The receiver coil array 122 is mounted on or close to a distortion source, such as the X-ray source 125 or the X-ray detector 135 of the fluoroscope 105. The transmitter coil array 115 is the movable assembly of the tracking system 110, and will thus be generally positioned remotely from the distortion source. The electromagnetic tracking system 110 measures and models mutual inductance between the transmitter coil array 115 and the receiver coil array 122. The mutual inductance is given by the ratio of the rate of change of current in the transmitter coil array 115 and the induced voltage in the receiver coil array 122.

The transmitter coil array 115 and the receiver coil array 122 are connected to a signal measuring circuit 155 that detects the levels of transmitter drive signals and the received signals, ratiometrically combining them to form a matrix representative of the mutual inductance of each of the pairs of component coils. The mutual inductance information, providing functions of the relative positions and orientations of the transmitter coil array 115 and the receiver coil array 122, is then processed by the processor 160 to determine corresponding coordinates.

In another embodiment, a method for electromagnetic (EM) tracking of position and orientation that utilizes a combination of discretized numerical field model and ring model to compensate for EM field distortion is provided. The discretized numerical field model is representation of spatially continuous EM field by a finite series of numerical field values.

The electromagnetic tracking system 110 focuses on creating a numerical model by either measuring or calculating the mutual inductance matrix over a sampled space. More specifically, for a given distortion source, a robotic arm is used to move the transmitter coil array 115 to different nodes of a pre-specified sampling grid to record the distorted data with respect to the receiver coil array 120, 121 or 122, which is rigidly attached to the distortion source. It is noted that the transmitter coil array 115 and the receiver coil array 120, 121 or 122 are interchangeable according to the theory of reciprocity.

The mutual inductance matrix and all related computation are conducted in the coordinate system defined by the receiver coil array 122. The corresponding undistorted P&O of the transmitter coil array 1 15 is also acquired in the receiver coordinates for each robot position by removing the distorters such as the X-ray source 125, the X-ray detector 135, and the C-arm 130 from the proximity of the receiver coil array 122.

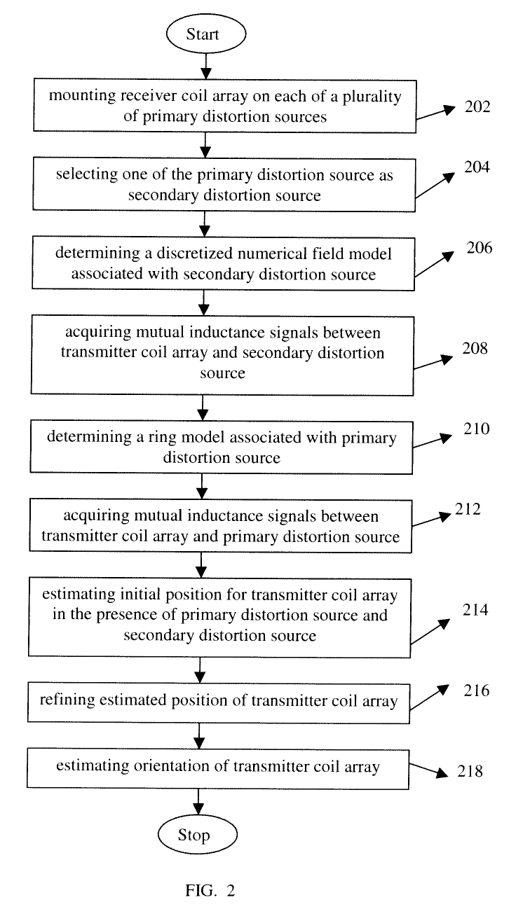

FIG. 2 shows one method 200 for collecting measurements for construction of a discretized numerical field model. The method 200 is performed by one or more of the various components of a robot enabled data collection system and process. Furthermore, the method 200 may be performed in software, hardware, or a combination thereof.

At 202, at least one receiver coil array 122 is mounted on each of a plurality of primary distortion sources, each of the primary distortion source comprising one of the X-ray source 125, the C-arm 130, the X-ray detector 135, the surgical table 145, the surgical tool 150, or other surgical instrument. At 204 one of the primary distortion source 125, 130, 135, 145 and 150 is selected as a secondary distortion source 145, at 206 a discretized numerical field model associated with the secondary distortion source 145 is determined, at 208 mutual inductance signals between the transmitter coil array 115 and the secondary distortion source 145 is acquired, at 210 a ring model associated with at least one primary distortion source 125, 130, 135 and 150 is determined, at 212 mutual inductance signals between the transmitter coil array 115 and the at least one primary distortion source 125, 130, 135 and 150 is acquired, at 214 an initial position for the surgical tool 150 in the presence of the primary distortion source 125, 130, 135 and 150 and the secondary distortion source 145 is estimated, at 216 the estimated position of the surgical tool 150 is refined and at 218 an orientation of the surgical tool 150 is estimated. The method is repeated for each selection of the primary distortion source 125, 130, 135, 145 and 150 as a secondary distortion source.

Determining a discretized numerical field model includes several steps. Firstly, the receiver coil array 122 is attached onto a reference wall fixed relative to a robot coordinate system. The robot position is recorded as well as the undistorted P&O of the transmitter coil array 115 relative to the receiver coil array 122. Secondly, a distortion source is attached to the receiver coil array 122. The distortion source may be, for example, the X-ray source 125, the X-ray detector 135 or the fluoroscopy C-arm 130. In other implementations, the distortion source may be the patient support table 145 or microscope, etc. With the to-be-measured distortion in place, the robot position is recorded as well as the distorted mutual inductance signal. With the data collected, the tracking system 110 may calculate distorted signals coupled from each of the transmitter coil array 115 to multiple receiver coils in expression of mutual inductance. The mutual inductance measurement can be expressed in a n.times.n matrix format where each element represents signal coupling between n transmitter coils and n receiver coils, respectively. A look-up table may be created using the measured mutual inductance. The look-up table cross-references the undistorted P&O of the transmitter coil array 115 and the distorted mutual inductance. The above-described method is one example of acquiring discretized numerical field model for a secondary distortion source 145 by collecting and calculating data associated with the secondary distortion source 145. Skilled artisans shall however appreciate that other known methods of acquiring discretized numerical field model may also be employed and all such methods lie within the scope of the invention.

The method for electromagnetic P&O tracking using the discretized numerical field model further includes estimating a seed position for the transmitter coil array 115 attached to the patient anatomy within the presence of the same secondary distortion source 145 associated with the acquired discretized numerical field model. Subsequent to obtaining the mutual inductance measurement between the transmitter coil array 115 and the receiver coil array 122, the difference between the computed mutual inductance and the estimated mutual inductance to each node on a subset of sample nodes surrounding the position of the transmitter coil array 115 can be monitored. The seed position is the node in the map having the smallest mutual inductance difference.

For ISCA tracking system 110, however, this direct seed-searching approach may experience numerical instability issue if any of the coordinate values is close to zero. This can be avoided by mathematically rotating the coordinate system to move the position far from the axes, calculating the position of the transmitter coil array 115 in the rotated coordinate system, and then mathematically de-rotating the result back to the original coordinate.

At 216 of FIG. 2, the estimate of the position of the transmitter coil array 115 is refined. This may be accomplished using an iterative fitting approach to create a best fit of the measured mutual inductances to the estimated mutual inductances. The position of the transmitter coil array 115 is dynamically adjusted in every iteration until the difference (or GOE—Goodness-of-fit) between measured and estimated mutual inductance is within a predetermined limit.

At 218, an estimate of the orientation of the transmitter coil array 115 is determined. To restore the undistorted sensor orientation, it is desirable to know the position of the transmitter coil array 115, which is used for the mutual inductance mapping. The orientations of the transmitter coil array 115 are readily available from the P&O map of the transmitter coil array 115. Since the transmitter coil array 115 is rigidly attached to the robot arm 130 during data collection, its orientation is likely to remain same for all map nodes as the transmitter coil array 115 is moved around to different robot locations. Thus, an estimation for distorted orientation can be obtained

If sufficient accuracy in position and orientation estimates is not achieved, then these estimates may be further refined by actions of block 216. At 216 of FIG. 2, both position and orientation estimates are simultaneously refined by using a numerical fitter to best fit the measured mutual inductances to the estimated mutual inductances. Both position and orientation are dynamically adjusted for all iterations until the difference between measured and estimated data is within the predetermined limit.

The method 200 described herein, may be implemented in many ways, including (but not limited to) medical devices, medical systems, program modules, general- and special-purpose computing systems, network servers and equipment, dedicated electronics and hardware, and as part of one or more computer networks.

In another embodiment, in order to acquire the ring model associated with each of the primary distortion source 125, 130, 135, 145 and 150, the intra-operative imaging and tracking system 100 may employ a plurality of conductive shields, or a plurality of sheath structures, each conductive shield configured to fit about or contain one of the primary distortion source 125, 130, 135, 145 and 150. Each conductive sheath standardizes the magnetic field disturbance introduced by the corresponding primary distortion source 125, 130, 135, 145 and 150. In some instances the conductive sheath may be a metal cylinder, dimensioned to enclose the corresponding primary distortion source 125, 130, 135, 145 and 150.

In another embodiment, rather than simply introducing the conductive sheath to form a standardized disturbance, the processor 160 may model such a disturbance. For example, the processor 160 may model a plurality of conductive sheaths; each conductive sheath fitted about a single primary distortion source 125, 130, 135, 145 and 150 as a conductive ring or cylinder at that region (using the known dimensions and behavior characteristics of the sheet metal material). The estimated disturbance may then be added to the stored values of a map of the undisturbed electromagnetic field to form an enhanced field map, or may otherwise be applied to enhance accuracy of tracking determinations. The estimated field may also be used to provide a seed value for determining position and orientation coordinates. A fitting procedure then refines the initial value to enhance the accuracy of the P&O determination.

Considering the scenario where the receiver coil array 122 is tracking the transmitter coil array 115, the discretized numerical field model accurately removes the effects of the secondary distortion source 145 on which the receiver coil array 122 is mounted. Each of the primary distortion sources 125, 130, 135, and 150 are distant enough for the receiver coil array 122 that their distortion is small and thus the ring model is used to remove the effects of the primary distortion sources 125, 130, 135 and 150. Considering that a single distortion source 125 can act as the primary distortion source for the receiver coil arrays 121 and 122 mounted on other distortion sources 135 and 145 respectively, and as the secondary distortion source for the receiver coil array 120 on which the distortion source 125 is mounted, each distortion source 125, 130, 135, 145 and 150 in the operating environment is mapped both by a discretized numerical field model and a ring model.

Therefore, the discretized numerical field model is determined for each of the plurality of primary distortion sources 125, 130, 135, 145 and 150 by selecting one of them as the secondary distortion source. Thus, the method 200 is repeated for each of the primary distortion sources 125, 130, 135, 145 and 150 by selecting one of them as the secondary distortion source. For each selection of the secondary distortion source (for example, 125), the ring model is determined for each of the rest of the primary distortion sources 130, 135, 145 and 150.

Upon obtaining complete representation of mutual inductance for the entire space of interest, the ring model is replaced with the more-accurate discretized numerical field model in order to track the distorted P&O of the transmitter coil array 115 in the receiver coil array 122 reference system. By tracking the plurality of distortion sources 125, 130, 135, 145 and 150 in the operating environment, we can numerically correct the field distortion and obtain accurate tracking.

The system and method described herein provide increased tracking accuracy, increased image accuracy, comprehensive and tight integration of tracking into the X-ray system providing ease of use and faster procedures.

In various embodiments, system and method for tracking a medical device are described. However, the embodiments are not limited and may be implemented in connection with different applications. The application of the invention can be extended to other areas, For example, in cardiac applications such as in catheter or flexible endoscope for tracking the path of travel of the catheter tip, to facilitate laser eye surgery by tracking the eye movements, in evaluating rehabilitation progress by measuring finger movement, to align prostheses during arthroplasty procedures and further to provide a stylus input for a Personal Digital Assistant (PDA). The invention provides a broad concept of tracking a device in obscure environment, which can be adapted to track the position of items other than medical devices in a variety of applications. That is, a tracking system may be used in other settings where the position of an instrument in an environment is unable to be accurately determined by visual inspection. For example, tracking technology may be used in forensic or security applications. Retail stores may use tracking technology to prevent theft of merchandise. Tracking systems are also often used in virtual reality systems or simulators. Accordingly, the invention is not limited to a medical device. The design can be carried further and implemented in various forms and specifications.

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An intra-operative imaging and tracking system for guiding a surgical tool during a surgical procedure performed on a patient, comprising: a fluoroscope having an X-ray source; an X-ray detector and a support structure configured to support the X-ray source and the X-ray detector, the X-ray source and the X-ray detector being movable about the patient to generate a plurality of two-dimensional X-ray images of the patient from different views; a tracking system comprising a transmitter coil array configured to generate an electromagnetic field in an area of interest, the transmitter coil array being affixed to the surgical tool and at least one receiver coil array configured to generate a sensing signal in response to sensed electromagnetic field, the at least one receiver coil array being secured against movement relative to one of a plurality of primary distortion sources; a signal measuring circuit electrically coupled to the tracking system to measure generated and sensed signals to form a matrix representing mutual inductance between the transmitter coil array and the receiver coil array; a processor operative with the mutual inductance matrix and the X-ray images to determine coordinates of the transmitter coil array affixed to the surgical tool and position of the surgical tool relative to the patient.

2. The system of claim 1, wherein the primary distortion source is one of the X-ray source, the X-ray detector and the support structure.

3. A method for electromagnetic tracking, the method comprising: mounting at least one receiver coil array on each of a plurality of primary distortion sources; selecting one of the primary distortion source as a secondary distortion source; acquiring mutual inductance signals between a transmitter coil array and the secondary distortion source, the transmitter coil array being rigidly attached to a surgical tool; acquiring mutual inductance signals between the transmitter coil array and at least one primary distortion source; estimating an initial position for the surgical tool in the presence of the primary distortion source and the secondary distortion source; refining the estimated position of the surgical tool and estimating an orientation of the surgical tool.

4. The method of claim 3, further comprising simultaneously refining estimates of both position and orientation.

5. The method of claim 3, wherein the refining is performed iteratively.

6. The method of claim 3, wherein the estimating an initial position comprises direct seed-searching and refining results of the direct seed-searching.

7. The method of claim 3, wherein the primary distortion source comprises a C-arm of a fluoroscope, X-ray detector of the fluoroscope, X-ray source of the fluoroscope, a surgical table, surgical equipment, or other surgical instrument.

8. The method of claim 3, wherein the acquiring comprises determining a discretized numerical field model associated with the secondary distortion source.

9. The method of claim 8, wherein the determining comprises: measuring undistorted position and orientation of the transmitter coil array at multiple positions and orientations in a designated volume without the presence of the secondary distortion source; measuring distorted mutual inductance between the transmitter coil array and the receiver coil array at multiple positions and orientations in the same designated volume with the presence of the secondary distortion source; mapping the undistorted position and orientation of the transmitter coil array and the distorted mutual inductance between the transmitter coil array and the receiver coil array.

10. The method of claim 3, wherein the acquiring comprises determining a ring model associated with at least one primary distortion source.

11. The method of claim 10, wherein the determining comprises: measuring undistorted position and orientation of the transmitter coil array at multiple positions and orientations in a designated volume without the presence of the primary distortion source; measuring distorted mutual inductance between the transmitter coil array and the receiver coil array at multiple positions and orientations in the same designated volume with the presence of the primary distortion source; mapping the undistorted position and orientation of the transmitter coil array and the distorted mutual inductance between the transmitter coil array and the receiver coil array.

12. One or more computer-readable media having computer-executable instructions thereon that, when executed by a computer, perform a method for electromagnetic tracking, the method comprising: mounting at least one receiver coil on each of a plurality of primary distortion sources; selecting one of the primary distortion source as a secondary distortion source; acquiring mutual inductance signals between a transmitter coil array and the secondary distortion source, the transmitter coil array being rigidly attached to a surgical tool; acquiring mutual inductance signals between the transmitter coil array and the at least one primary distortion source; estimating an initial position for the surgical tool in the presence of the primary distortion source and the secondary distortion source; refining the estimated position of the surgical tool and estimating an orientation of the surgical tool.

13. The computer readable media of claim 12, further comprising simultaneously refining estimates of both position and orientation.

14. The computer readable media of claim 12, wherein the refining is performed iteratively.

15. The computer readable media of claim 12, wherein the primary distortion source comprises a C-arm of a fluoroscope, X-ray detector of the fluoroscope, X-ray source of the fluoroscope, a surgical table, surgical equipment, or other surgical instrument.

16. The computer readable media of claim 12, wherein the acquiring comprises determining a discretized numerical field model associated with the secondary distortion source.

17. The computer readable media of claim 16, wherein the determining comprises: measuring undistorted position and orientation of the transmitter coil array at multiple positions and orientations in a designated volume without the presence of the secondary distortion source; measuring distorted mutual inductance between the transmitter coil array and the receiver coil array at multiple positions and orientations in the same designated volume with the presence of the secondary distortion source; mapping the undistorted position of the transmitter coil array and the distorted mutual inductance between the transmitter coil array and the receiver coil array.

18. The computer readable media of claim 12, wherein the acquiring comprises determining a ring model associated with at least one primary distortion source.

19. The computer readable media of claim 18, wherein the determining comprises: measuring undistorted position and orientation of the transmitter coil array at multiple positions and orientations in an designated volume without the presence of the at least one primary distortion source; measuring distorted mutual inductance between the transmitter coil array and the receiver coil array at multiple positions and orientations in the same designated volume with the presence of the at least one distortion source; mapping the undistorted position of the transmitter coil array and the distorted mutual inductance between the transmitter coil array and the receiver coil array.
